(19) United States
(12) Patent Application Publication
(10) Pub. No.: US 2013/0261410 A1
(43) Pub. Date: Oct. 3, 2013

(54) System and Method for Body and In-Vivo Device, Motion and Orientation Sensing and Analysis

(71) Applicant: Roger Davenport, Fort Lauderdale, FL (US)

(72) Inventor: Roger Davenport, Fort Lauderdale, FL (US)

(73) Assignee: Larger Reality Technologies LLC, Fort Lauderdale, FL (US)

(21) Appl. No.: 13/432,862

(22) Filed: Mar. 28, 2012

(51) Int. Cl. A61B 5/07

(52) U.S. Cl. 600/302

(57) ABSTRACT

A measurement and analysis system for characterizing body core motional acceleration and orientation with respect to gravity, and characterizing the motional acceleration and orientation of an ingestible device relative to the body core with associated activity level, and under some conditions, characterizing the motional acceleration of digestive track lumens relative to the body core with associated activity level. The system comprises an in-vivo ingestible sensing device that travels through the digestive track and an external sensing device attached externally to the body core and each device measures orthogonal acceleration vectors data that are transmitted to a computation device that executes algorithms to determine all characterizations. The benefits of the measurement and analysis system are: 1.) increased understanding of digestive track behaviors, 2.) when combined with ingestible diagnostic systems provides increased accuracy through real time environmental knowledge.

### FIELD OF THE INVENTION

The present invention relates to a measure system and analytical method for determining the motional characteristics and dynamics orientation characteristics or tumbling characteristics of an in-vivo or ingested sensing device(s) relative to the body core of a human or animal. Further, the invention relates to provides characterization of the body core activities and orientation the body core to a gravitation field that strongly affect the behavior of the ingested sensing device.

### BACKGROUND OF THE INVENTION

There is a need for a greater understanding of the motility and more specifically movement and dynamic orientation of an object in the digestive track relative to the a host body core with activity level defined and further, movement characterization digestive track lumens relative to the a host body core with activity level defined. The benefits of such a system are multifaceted, 1.) a more accurate characterization of the digestive track while the body is involved with different activities, 2.) increased effectiveness of ingestible diagnostic systems taking ingestible device tumbling into account.

There are many methods for determine motility of objects in the digestive track, however, these methods do not address the dynamic orientation characteristics of the object moving through the digestive track and motional characterization of the digestive track lumens relative to the body core, and body core movement relative to a gravitation field vector. This invention provides in combination all of these characterization through the combined use of two separate acceleration measurement devices: an ingestible three dimensional acceleration sensor pill, and an externally attached three dimensional acceleration sensor patch.

The approach used in prior art include in-vivo diagnostic devices that move through the GI tract and collect data and transmit the data to a receiver system. Further, there are many examples of prior art that teach use imaging technologies such as optical imaging means to determine motility and event orientation changes of objects in digestive track. However these methods fail to teach a system that can provides detailed dynamic orientation changes of object as they travel through the digestive track relative to lumens in combination with movement characterization of the lumens relative to the body core and further movement and orientation of the body core relative to the vector produced by gravity.

An example of a system is U.S. Pat. No. 7,724,928 to Glukhovsky et al. a device system and method for motility measurements. The ingestible device may include a three dimensional accelerometer. Glukhover teaches using integration on the three dimensional accelerometer signal to determine motility. Glukhover's system does not provides for a second measurement device located on the body core surface that measures three dimensional acceleration. Further, Glukhovsky does not teach and cannot provide a separation of the motional and dynamic orientation characterizations of the ingestible device relative to the body core.

Another example of a system is U.S. Pat. No. 7,200,253 to Glukhovsky et al. teaches a system and method for measuring and analyzing the motility within the body lumen such as the gastrointestinal track where an in vivo imaging device such as a capsule captures images and transmits the images to a processor, which calculates the motility of the device based on comparison of the images. Glukhovsky fails to teach a method of using multiple acceleration measurement devices including at least one ingestible pill with acceleration measurement capabilities and one external acceleration measurement patch to characterize the following combination attributes: object tumbling with the digestive relative to the digestive track, motional characterization of the digestive track lumens relative to the body core, and body core movement relative to a gravitation field vector.

Another example of such as system is U.S. Pat. No. 7,833,151 to Khait et al. that teaches an ingestible diagnostic pill that comprises of two imagers on each end of the pill. The art teaches that the two imagers provides improved imaging coverage for instances where the pill tumbles or changes orientation in the lumens. The result of moving to two imagers is a solution to the situation of a single imaging pills changing orientation and not providing enough image coverage of the lumens interior surface. In conclusion, a camera pill can detect orientation changes however, because of the slow imaging rate a full characterization of dynamic and granular orientation changes are not possible. Khait fails to teach a method of using multiple acceleration measurement devices including at least one ingestible pill with acceleration measurement capabilities and one external acceleration measurement patch to characterize the following combination: object tumbling with the digestive relative to the body core, motional characterization of the digestive track lumens relative to the body core, and body core movement relative to a gravitation field vector.

Another example of a system is Pub. No. US 2009/0182207 A1 to Risky et al. teaches an ingestible bolus configured to be maintained in a stomach of an animal may be formed from a substantially cylindrical enclosure shell. The bolus contains an accelerometer sensor may be a three-axis accelerometer and may be configured to detect animal movement characteristics and animal stomach contractions as a single inseparable measurement. Risky does not teach a system that comprises two acceleration measurements devices one internal to the digestive track and one externally attached to the animal body that would allow for the separation of body movement relative to space and bolus movement relative to the body core.

### BRIEF SUMMARY OF INVENTION

The present invention is a measurement and diagnostic system that provides detailed characterizations of movements and orientation of: 1.) the core body that encompasses the digestive track, and 2.) provides detailed characterization of movements of the of portions of the digestive track lumens relative to the host body core, and 3.) provides detailed characterization of the dynamic orientation of an object within the lumens of the digestive track relative to the digestive track. The diagnostic system is applicable for humans and animals.

The system comprises one or more in-vivo or ingestible acceleration sensor pill, one or more external body surface acceleration sensor patch(es), a data transport and synchronization sub-system and a wireless data reception and processing and display sub-system device that further comprises software to implement intelligent algorithms for the analysis of the multiple sources of synchronized measurement data.

The ingestible sensor pill comprises a three dimensional accelerometer in electrical connection with electronics that provide the functions of: data capture of the acceleration measurements, memory storage of the measured data, wireless communications for exporting the measured data and an energy source such as a battery to operate the electronics. Further the three dimensional accelerometer is rigidly mounted in the pill housing to define a pill measurement coordinate system that is in a fixed relationship to the ingestible sensor pill housing. The external body surface sensor patch comprises a three dimensional accelerometer in electrical connection with electronics that provide the functions of: data capture of the acceleration measurements, memory storage of the measured data, wireless communications for exporting the measured data and an energy source such as a battery to operate the electronics. Further the three dimensional accelerometer is rigidly mounted in the patch housing to define a patch measurement coordinate system. The patch housing is further attached or secured externally to the body core in close proximity to the digestive track where minimal relative movement will occur between the skeletal frame of the body and patch housing. An example location for a human body may be the lower part of the back near the spine that is in close proximity to the digestive track. Also the patch attachment is aligned to the body core with a predetermined orientation creating a substantially fixed relationship between the body core and the patch measurement coordinate system.

The analysis algorithms to process the acceleration measurement data from the ingestible acceleration sensing pill device and the acceleration measurement data from the external body surface acceleration sensor patch. The analysis algorithm use vector analysis of the two separate measurement devices each providing three dimensional acceleration measurements vector results defined on their respective measurement coordinate system. The measurement results are processed and analyzed first separately and then in combination through additive and or differential vector analysis to determine the three categories of motion and or orientation results previously mentioned. This is possible with the known common acceleration force vector component of gravity affecting each measurement device and their individual vector measurements components due to motional acceleration that is individual to each measurement device coordinate system.

The measurement and analysis system provides motional and orientation characterizations that include:
1. Characterization of body core movement and orientation such a stationary vertical or stationary laying down or variations in between with associated:
   a. Gastrointestinal dynamic lumen movement relative to the body core
   b. Pill dynamic orientation or tumbling relative to the body core and or selected gastrointestinal lumen
2. Characterization of body core movement and orientation such a physical activity such as walking and running with associated:
   a. Gastrointestinal dynamic lumen movement relative to the body core
   b. Pill dynamic orientation or tumbling relative to the body core and or gastrointestinal lumens
3. Statistical analysis of pill orientation relative to body core for a predefined body activity that consists of:
   a. Average rate of pill orientation change in degrees/sec over a predetermined time period
   b. Peak rate of pill orientation change in degrees/sec over a predetermined time period
4. Results analysis of the previous capabilities 1, 2 and 3 specific to a given section of the gastrointestinal track, such as esophagus, stomach, small intestines, large intestines and colon.
5. All above results can be conducted with different size, weight and shape of ingestible diagnostics pills.

The system can also be used with or integrated with other ingestible diagnostic sensor type systems that can be improved with real time knowledge of diagnostic pill orientation. An embodiment of the measurement motion, orientation and analysis system is the integration of conventional diagnostics sensors such as optical imaging into the ingestible acceleration sensor pill that would allow real time orientation and analysis information to be provided to the imaging system enabling a more efficient use of imaging rate with respect to the pill ability to capture 100% of the lumens wall surface with fewer images required based on tumbling rates.

### BRIEF DESCRIPTION OF DRAWINGS

The above and other features of the present invention will become more apparent upon reading the following detailed description in conjunction with the accompanying drawings, in which:

FIG. 1 is a system view of the present invention showing the ingestible sensor pill 101 within the intestinal track, the external sensor patch 102 attached to the surface of the body core and a data processing user interface device 103.

FIG. 2 shows the ingestible sensor pill 101 comprising the measurement and electronic functions within a housing.

FIG. 2A shows the ingestible sensor pill housing and the pill measurement coordinate system.

FIG. 3 shows the sensor patch 102 comprising the measurement and electronic functions within a housing.

FIG. 3A shows the sensor patch 102 and associated measurement coordinate system

FIG. 3B shows the sensor patch 102 attached to the body core 305 of a human body with an exemplary predetermined location and alignment of the sensor patch and sensor patch measurement coordinate system referenced to the body core features.

FIG. 4 shows the data processing user interface device 103 comprising electronics functions for the reception and processing of measurement data.

FIG. 5 shows an exemplary sensor patch acceleration measurement vector 510 \vec{a_{patch}} and it's vector components resulting from; gravity 501 \vec{a_{g}} and body core movement relative to gravity 501 \vec{a_{bc(g)}}

FIG. 5A shows an exemplary sensor pill acceleration measurement vector 511 \vec{aₚᵢₗₗ} and it's vector components resulting from; gravity 501 \vec{a_{g}} and body core motional acceleration component relative to gravity 501 \vec{a_{bc(g)}} and the sensor pill motional acceleration component relative to the body core that is further relative to gravity 503 \vec{a_{pill(bc(g))}}.

FIG. 6 show the sensor patch 102 and measurement coordinate system 601 with an exemplary orientation with an exemplary sensor patch acceleration measurement vector 510 {right arrow over (\vec{a_{patch}} )}.

FIG. 6A show the sensor pill 101 and measurement coordinate system 602 with an exemplary orientation with an exemplary sensor pill acceleration measurement vector 511 \vec{aₚᵢₗₗ}.

FIG. 6B show the gravitational acceleration component from the patch 5101 and the gravitational acceleration component from the pill 5111 applied to a single coordinate system to determine the orientation of the pill relative to the patch.

FIG. 7 show a common coordinate system with exemplary sensor pill and sensor patch measurement vectors (\vec{aₚᵢₗₗ}, \vec{a_{patch}}) and each measurement vector's acceleration component vectors.

FIG. 7A shows a common coordinate system with exemplary sensor patch measurement vector \vec{a_{patch}} and sensor patch measurement vector rotated \vec{aₚᵢₗₗ₋ᵣₒₜ} by the derived orientation relationship between the sensor pill and sensor patch, for the calculation of the acceleration component \vec{a_{pill-rot(bc(g))}} representing the sensor pill motional relative to the body core that is further relative to the gravitational vector.

### DETAILED DESCRIPTION OF INVENTION

The present invention is a measurement and diagnostic analysis system to characterize the dynamic motion and orientation of the body core and further characterizes dynamic motion and orientation of an object within the digestive track relative to the body core and its associated physical activities. Under certain conditions the system also characterizes lumen movement relative to the body core and its associated physical activities.

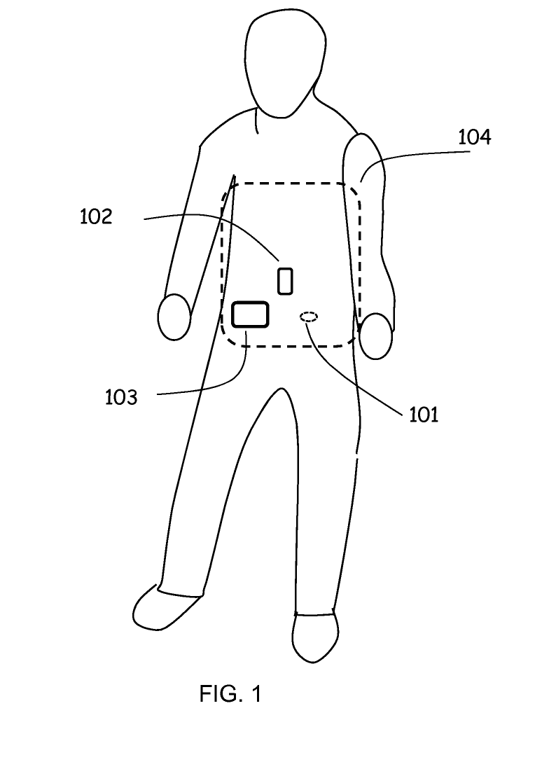

As shown in FIG. 1 the preferred embodiment of the measurement and motional and dynamic orientation characterization system comprises three types of devices: one or more in-vivo ingestible sensor pill(s) 101 that travel through the digestive track with wireless communications abilities and one or more attachable sensor patch(s) 102 that attach to the external surface of the body core 104 with wireless communications abilities, and a data processing user interface device 103 with wireless communication abilities that is carried externally to the body core. During system operation the sensor pill 101 travels through the digestive track and continuously measuring acceleration in three dimensions and wirelessly transmits the acceleration measurement data to the data processing user interface device 103. Simultaneously the sensor patch 102 is measuring acceleration in three dimensions and wirelessly transmits the synchronized acceleration measurement data to the data processing user interface device 103. The sensor patch 102 is attached or securely held in place with a predetermined location on the surface of the body core 104 and with a predetermined orientation to the body core. The data processing user interface device 103 receives the synchronized measurement data from the sensor pill 101 and the sensor patch 102 and uses algorithms to process all measured data to determine in virtual real time the dynamic motion and orientation characterization for: body core dynamic motion and dynamic orientation relative to gravity, sensor pill dynamic motion and dynamic orientation relative to the body core, and under certain conditions the dynamic motion of lumens of the digestive track relative to the body core.

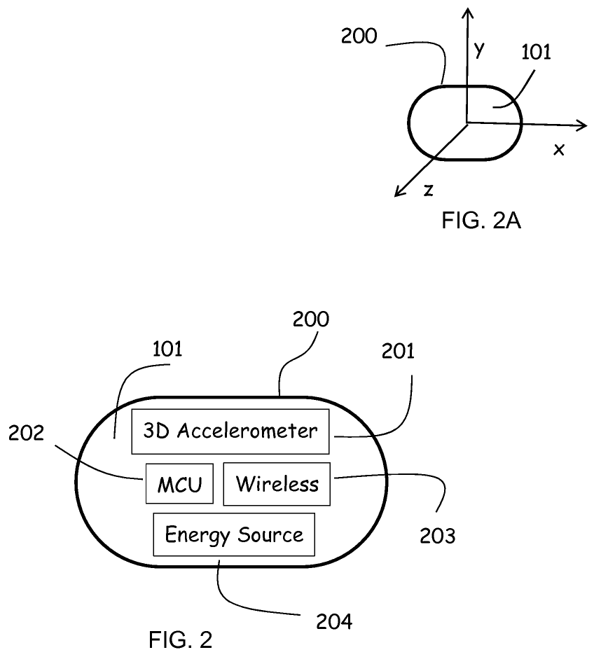

As shown in FIG. 2 the sensor pill 101 comprises a housing 200, a three dimensional accelerometer 201 in electrical connection with a micro controller unit (MCU) 202 in electrical connection with a wireless communication section 203. Further all electronic circuitry are in electrical connection with an energy source 204 such as battery or capacitive device(s). The housing 200 has a size, shape and weight that can be any predefined size, shape and weight as desired as long as it can contain the required electronics and is made of standard material(s) that are approved by the FDA for an ingestible medical diagnostic device. Various sizes, shapes and weights may be desired as a controlled input variable for motional and dynamics orientation characterization of said sensor pill 101 in the digestive track. The micro controller unit 202 comprises standard electronic functions for acceleration sensor sampling and data capture, memory storage, control and synchronization of other peripheral circuitry. The sampling rate of the acceleration sensor 201 signal by the MCU is set to the Nyquest sampling rate of the highest pertinent frequency component of any of three acceleration measurement axes signals from either the pill's accelerometer 201 or the patch's accelerometer (not shown) to ensure no information is lost to aliasing. The wireless communication section 203 in the preferred embodiment uses RF communication, however, in alternate embodiments the wireless communication section may utilize acoustic, magnetic or any other of wireless signal that can propagate a signal or field through human or animal tissue. In the preferred embodiment the wireless RF communication section 203 can be implemented with standard off the shelf RF integrated circuits that can implement a variety of standard protocols such as BlueTooth™, Zigbee™, WiFi, WiMax™ or any other standards based protocol. The RF communication section 203 can also be implemented with non-standard protocols that are programed and designed with standard off the shelf agnostic RF circuitry to customize data rates and bandwidth usage. The RF section also contains an antenna designed for the selected frequencies of operation. The weight, size and shape of the pill 101 can be designed to varies specifications for evaluation of motility, motion and orientation characterization with different physical attributes. For example the sensor pill 101 may be round, oval, rectangular or any other shape with varied weight and balance.

As shown in FIG. 2B the sensor pill 101 has a fixed three dimensional measurement coordinate system with axes x, y and z that are a result of the fixed predetermined mounting location and predetermined orientation of the accelerometer within the housing 200. The predetermined accelerometer 201 mounting location (not shown) is preferably at the center of gravity of the pill, however the sensor 201 mounting may be anywhere within the housing. The predetermined orientation of the acceleration sensor 201 is defined to align specific axes of the measurement coordinate system with known shape features of the pill. The measurement axes of the accelerometer in this fixed predetermined relationship to the housing and defines the sensor pill's measurement coordinate system.

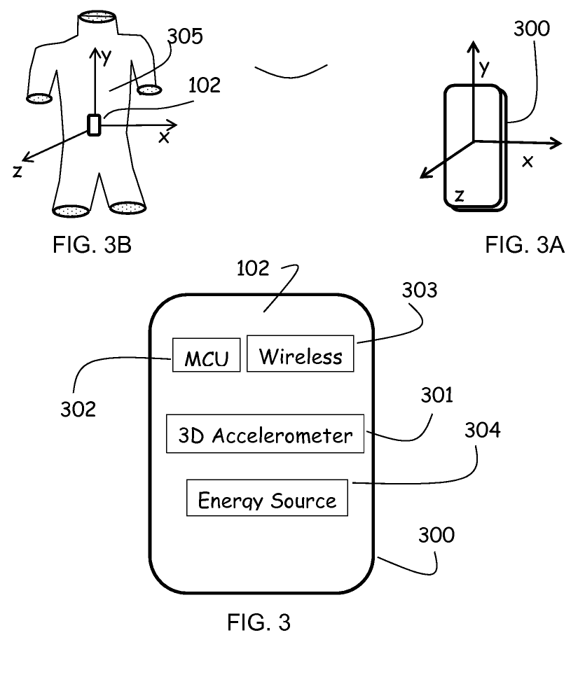

As shown in FIG. 3 the sensor patch 102 comprises a housing 300, a three dimensional accelerometer 301, in electrical connection with a micro controller unit (MCU) 302 in electrical connection with a wireless communication section 303. Further all electronic circuitry are in electrical connection with an energy source 304 such as battery or capacitive device(s). The housing 300 can be implemented with a plastic or other lightweight material. The three dimensional accelerometer 301 may be implement with a standard MEMS device three dimensional accelerometer. The accelerometer 301 is rigidly mounted within the housing 300 to provides the predetermined three dimensional measurement axes x y and z of the sensor patches measurement coordinate system as shown in FIG. 3A. The micro controller unit 302 comprises standard electronic functions for sensor signal sampling and data capture, memory storage, control and synchronization of other peripheral circuitry. The sampling rate of the acceleration sensor device 301 signal by the MCU 302 is set to the Nyquest sampling rate of the highest pertinent frequency component of either pill accelerometer 201(not shown in FIG. 3) or the patch accelerometer 301 measurement axes signals to ensure no information is lost to aliasing. The wireless communication section 303 in the preferred embodiment uses RF communications, however, in alternate embodiments the wireless communication section may implement acoustic, magnetic or any other of wireless signal that can propagate a signal or field through human or animal tissue. In the preferred embodiment the wireless RF communication section 303 can be implement with standard off the shelf RF integrated circuits that can be used for a variety of standard protocols such as BlueTooth™, Zigbee™, WiFi, WiMax™ or any other standards based protocol. The RF section can also be implemented with non-standard protocols that are programed and designed with standard off the shelf agnostic RF circuitry to customize data rates and bandwidth usage. The RF section 303 also contains an antenna that is designed for the selected frequencies of operation.

As shown 3B the sensor patch 102 is attached externally to the body core 305. The sensor patch 102 is placed at a predetermined location with a predetermined orientation relative to the body core 305. For humans an exemplary predetermined location for the sensor patch 102 is on the surface of the lower back of the body core 305 on or near the spine. This allows the sensor patch 102 to be in close proximity to the intestinal track on a portion of the body core that has less fat and therefore less movement variables due to fat movement and jiggling in relation to the skeletal structure of the body core as measured by the acceleration measurement device 301 of the sensor patch 102. Further, the preferred orientation of the sensor patch 102 aligns the y axis of the sensor patch coordinate system with the spine or in near alignment with the spine and aligns the x axis with a horizontal line parallel or near parallel to line between the hips of the of the body core when the body core 305 is standing upright and the z axis is aligned perpendicular or near perpendicular to the general plain of the back of the body core 305. In alternate embodiments the sensor patch 102 can have a predetermined location and orientation that vary, however, as long as location and orientation are known, all characterization can be accomplished. The attachment mechanism (not shown) for the sensor patch 102 can be a medical adhesive, a medical tape or a strap that is adapted to hold the sensor patch in a predetermined location with a predetermined orientation to the body core 305.

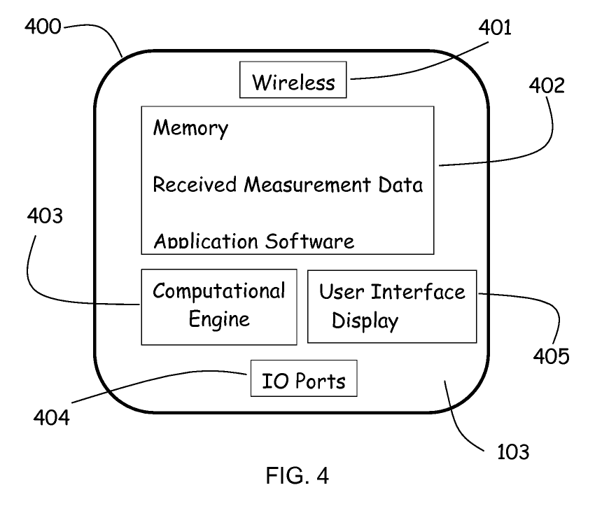

As shown in FIG. 4 the data processing user interface device 103 comprises a housing 400 and the housing 400 further comprises electronics that provides the functions of wireless communications 401 to receive measured data from the sensor pill 101 and sensor patch 102, a digital memory 402 for storing measurement data and for storing application software the contains algorithms for the interpretation of the sensors measurement data, a computation engine 403 for executing the application software algorithms, conventional IO ports 404 such USB or IEEE1394 or others for the transfer of raw measurement data or the transfer calculated motional and orientation result to other common types of computer based devices. The data processing user interface device 103 may also have a user interface screen 405 for direct user interfacing and results viewing. The data processing user interface device 103 may be an off the shelf device such as a laptop computer, a netbook, a Pad or Tablet, a smart phone or any other common processor based device with the above mentioned functional attributes. The data processing user interface device 103 may also be a custom device designed with the above functionality with off the shelf components and modules.

Algorithms that are part of the application software interpret the synchronized acceleration measured data. The algorithms are based on vector analysis and magnitude analysis of the combination of measured data from both the sensor pill 101 and the sensor patch 102 over a period of time producing a time line result of motional characteristics and dynamics orientation characteristics. To understand the basis of the analysis, first a summary of contributing forces to the acceleration measurement results are reviewed for the sensor patch 102 and the sensor pill 101 separately.

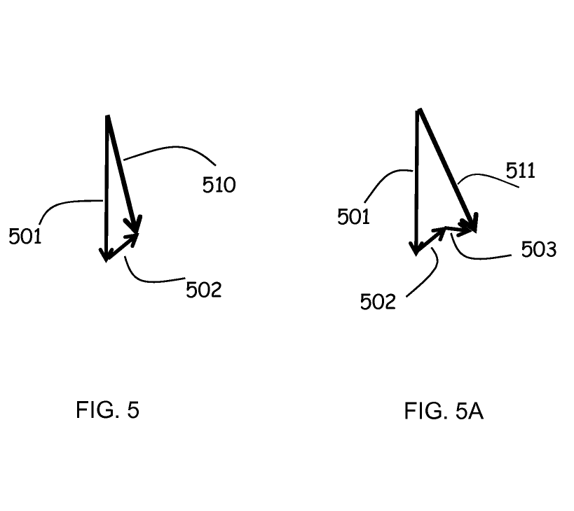

As shown in FIG. 5 the acceleration force components (in an exemplary vector configuration) that act on, and are measured by, the sensor patch 102 include: the gravitational acceleration force component 501 \vec{a_{g}} and the body core motional acceleration component 502 \vec{a_{bc(g)}} relative to gravity. These two acceleration vectors components add to create the sensor patch measurement vector 510 \vec{a_{patch}}.

\vec{a_{patch}}=\vec{a_{g}}+\vec{a_{bc(g)}}

As shown in FIG. 5A the acceleration force components in an exemplary configuration that act on, and are measured by, the sensor pill 101 include: the gravitational acceleration force component 501 \vec{a_{g}} and three motional acceleration components. The three subcomponents of motion in terms of acceleration include: the body core motion acceleration component 502 \vec{a_{bc(g)}} relative to gravity, and the sensor pill motion acceleration component 503 \vec{a_{pill(bc(g))}} relative to the body core that is relative to gravity. These acceleration components all add to create the single sensor pill measurement vector 511 \vec{aₚᵢₗₗ}.

\vec{aₚᵢₗₗ}=\vec{a_{g}}+\vec{a_{bc(g)}}+\vec{a_{pill(bc(g))}}

Starting from these two equations all of the various characterizations of translational motion and orientation can be calculated for the body core, and the sensor pill relative to the body core. In summary the analysis methods will be presented in the following order,
1. analysis of dynamic body core orientation and dynamics motion resulting from spatial translational movement relative to the gravitation field
2. analysis of ingestible object or sensor pill dynamic motion and dynamic orientation or tumbling (with known body core activity level) relative to:
   a. body core
   b. gravitational field
3. analysis of lumens motion (with known body core activity level) relative to:
   a. body core for smaller lumen such as the small intestines
   b. gravitational field for smaller lumen such as the small intestines

The above analytical results are based on sensor pill 101 and sensor patch 102 measured data and know environmental forces and defined physical relationships that include:
1. The gravitation vector force (1g)
2. The patch measurement coordinate system location and orientation relative to the body core
3. The body core is always in the gravitational field
4. The pill measurement coordinate system can have any orientation to the body core

The first analytical characterization defines the condition of body core 305 with respect to: activity represented by a motional characterization and orientation relationship angle relative to the gravitational force vector.

For the condition where the body core is static or moving slowly, the motional acceleration component for the body core 502 \vec{a_{bc(g)}} relative to gravity is zero or very small compared to gravitational acceleration force vector \vec{a_{g}}. Therefore the sensor patch 102 measured vector 510 \vec{a_{patch}} is simply equal to the gravitational acceleration force vector \vec{a_{g}}

\vec{a_{patch}}=\vec{a_{g}}

Therefore, orientation of the sensor patch (and body core) with respect to the gravitation field vector (for the preferred embodiment patch predetermined orientation to body core) is equal to the angle of between the y axes of the sensor patch measurement coordinate system and the sensor patch measurement vector \vec{a_{patch}} that is equal to the gravitation force vector \vec{a_{g}}. When measurements are taken over a time period, the dynamic orientation of the body core relative to the gravitation field vector is represented on a time line. This type of characterization may apply to activities levels of standing, sitting or lying down.

For the condition where the body activity involves moving more rigorously, acceleration signature traits such as those for walking or running that are used by pedometers can be employed to identify the activity type as measured by the sensor \vec{a_{patch}} vector measurement. Since walking, jogging or running are spatial translational activities with a repetitive and cyclical movement nature and have a generally constant velocity over time the general orientation of the body core to the gravitation field can be achieved with averaging the sensor patch measurement vector over a time span equal to one or more cycles of the cyclical movement.

AVG[\vec{a_{patch}}]=\vec{a_{g}}

The general orientation of the body core relative to the gravitational field during rigorous activity can be solved as the angle between the AVG[\vec{a_{patch}}] vector and the y axis of the sensor patch 102 patch measurement coordinate system. Further, with the acceleration component due to the gravitation field defined, the body core acceleration component \vec{a_{bc(g)}} is defined by:

\vec{a_{bc(g)}}=\vec{a_{patch}}−AVG[\vec{a_{patch}}]

Next, to characterize and provide analysis of the ingestible object or sensor pill dynamic orientation or tumbling and dynamic motion relative to the body core, measurement data from both sensor pill 101 and sensor patch 102 are required.

To characterize and provide analysis of the dynamic orientation or tumbling of the pill relative to the body core that is further relative to the gravitation force vector, the dominant known acceleration component of gravity \vec{a_{g}} is used as it is applied to each of the independent measurement coordinate systems of the sensor pill 101 and sensor patch 102. Using the known dominant gravitational acceleration vector \vec{a_{g}}, the relative angle of rotation between the two measurement coordinate systems can be evaluated.

As previously described, when the body core is in a low activity condition, the sensor patch 102 measurement vector 510 \vec{a_{patch}} is equal to the acceleration component of gravity \vec{a_{g}}. Further, for the body core active with a cyclical motional activity such as jogging, the sensor patch measurement vector 510 \vec{a_{patch}} can be averaged over a time duration equal to one or more cycles of the repetitive activity to extract the gravitation acceleration component \vec{a_{g}} vector as defined on the sensor patch 102 measurement coordinate system.

The same methodology can be applied to the sensor pill 101 measurement vector to extract the gravitation acceleration component \vec{a_{g}} defined on the sensor pill measurement coordinate system. When the body core is in a low activity condition, the sensor pill 101 measurement vector 511 \vec{aₚᵢₗₗ} is equal to the acceleration component of gravity and a motional component due to pill movement due to peristalsis in the digestive track. However, the magnitude of the motional vector component due to peristalsis is less than 1/300 as compared to the magnitude of the acceleration component due to gravity and therefore can be ignored. When the body core activity is rigorous such as when running or jogging, time averaging can be applied to the sensor time varying sensor pill 101 measurement vector \vec{aₚᵢₗₗ} to extract the time varying acceleration gravitation component as defined on the sensor pill 101 measurement coordinate system.

Using the acceleration component of the gravitational vector as measured on the two independent measurement coordinate systems, the angles of rotation of the sensor pill 101 measurement coordinate system relative to the sensor patch 102 measurement coordinate system can be defined.

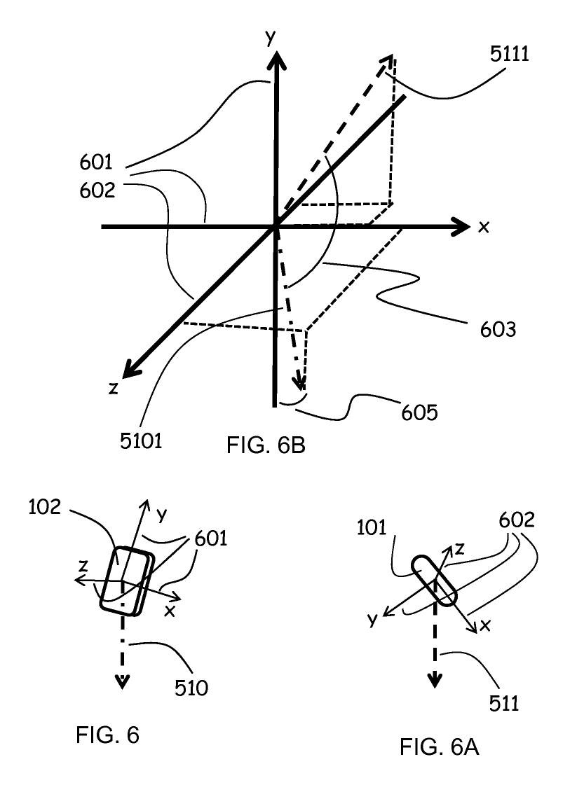

As shown in FIG. 6 the sensor patch 102 at a given instant in time has a given sensor patch 102 measured vector 510 \vec{a_{patch}} that is defined on the sensor patch measurement coordinate system axes 601. Further, from sensor patch 102 measured vector 510 \vec{a_{patch}}, the acceleration component due to gravity 501 \vec{a_{g-patch}} (Shown in FIG. 5) can be extracted referenced to the sensor patch measurement coordinate system 601 with x y and z axes unit vectors (u_{x-patch-mcs}) and (u_{y-patch-mcs}) and (u_{z-patch-mcs})

\vec{a_{g-patch}}=a_{xg-patch}(u_{x-patch-mcs})+a_{yg-patch}(u_{y-patch-mcs})+a_{zg-patch}(u_{z-patch-mcs})

Also, shown in the FIG. 6A the sensor pill 101 measured vector 511 \vec{aₚᵢₗₗ} at the same instant in time is defined on the sensor pill 101 measurement coordinate system axes 602. Further the acceleration component due to gravity 501 \vec{a_{g-pill}} (FIG. 5A) can be extracted from sensor pill 101 measured vector 511 \vec{aₚᵢₗₗ} referenced to the sensor pill measurement coordinate system 602 with x y and z axes unit vectors (u_{x-pill-mcs}) and (u_{y-pill-mcs}) and (u_{z-pill-mcs}).

\vec{a_{g-pill}}=a_{xg-pill}(u_{x-pill-mcs})+a_{yg-pill}(u_{y-pill-mcs})+a_{zg-pill}(u_{z-pill-mcs})

The orientation relationship of the two measurement coordinate systems to one another is a variable compound angle of rotation that defines the sensor pill's orientation relative to the sensor patches orientation. The common gravitation acceleration component that is applied to the two individual measurement coordinate systems with random relative orientation is used to define the orientation relationship between the measurement coordinate system at a given point in time.

As show in FIG. 6B when the sensor pill measurement coordinate system 602 is rotated to align it's x,y and z axes to the sensor patch measurement coordinate system 601 x, y and z axes, the compound angle of orientation 603 of the sensor pill 101 gravitational acceleration component \vec{a_{g-pill}} 5111 relative to the sensor patch 102 gravitational acceleration component \vec{a_{g-patch}} 5101 is defined and is also the orientation of the pill 101 relative to the patch 102 and body core. Further, as earlier described in the body core activity analysis using the sensor patch 102 gravitational component \vec{a_{g-patch}} and the preferred embodiment of predetermine patch attachment location and orientation to the body core, the compound angle of orientation 605 of the body core relative to the gravitation field is defined. All relative orientation results when describe in a three dimensional Cartesian space are compound angle requiring two angles of rotation to completely describe the orientation relationship as defined on a single coordinate system.

Now to solve the for the dynamic motion of the pill relative to the body core we must define the last unknown acceleration motional component 503 \vec{a_{pill(bc(g))}}; of the sensor pill 101 measurement vector 511 \vec{aₚᵢₗₗ}. To do this first, a summary of what has been solved and is known at this point in the analysis methodology:
1. From the sensor patch measurement vector \vec{a_{patch}} the variables that have been solved are:
   a. the acceleration component due to gravity \vec{a_{g-patch}} as measured on sensor patch coordinate system
   b. the acceleration component due to spatial translations motions of the body core relative to gravitational vector \vec{a_{bc(g)}} as measured on the sensor patch measurement coordinate system
2. From the sensor pill measurement vector \vec{aₚᵢₗₗ} the variables that have been solved are:
   a. the acceleration component due to gravity \vec{a_{g-pill}} as measured on sensor pill measurement coordinate system
3. From the acceleration component due to gravity \vec{a_{g-patch}} as measured on sensor patch coordinate system and the acceleration component due to gravity \vec{a_{g-pill}} as measured on sensor pill measurement coordinate system, the variables that have been solved are:
   a. The compound angle of orientation 603 that describes the orientation relationship between the sensor pill and the sensor patch
4. The magnitude of the acceleration components \vec{a_{g-pill}} and \vec{a_{g-patch}} are equal.
5. The magnitude of the acceleration component \vec{a_{bc(g)}} that is applied to the pill or patch measurement coordinate system is equal, and further, the acceleration component \vec{a_{bc(g)}} orientation relationship relative to the respective gravitational acceleration component is equal.

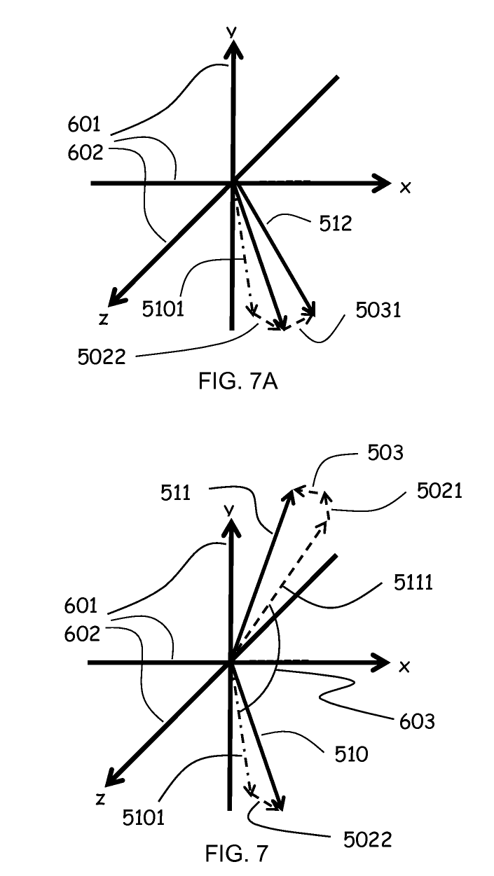

As shown in FIG. 7. The measurement coordinate systems 601 for the patch and 602 for the pill are aligned to create a single common measurement coordinate system. On the common coordinate system the sensor pill measurement vector 511 \vec{aₚᵢₗₗ} is shown with its acceleration component vectors due to: gravity 5111 \vec{a_{g-pill}}, motion of the body core relative to gravity 5021 \vec{a_{bc(g)}} and the motion of the pill relative to the body core relative to gravity. Also shown on the common measurement coordinate system is the sensor patch measurement vector 510 \vec{a_{patch}} with its acceleration component vectors due to: gravity 5101 \vec{a_{g-patch}} and motion of the body core relative to gravity 5022 \vec{a_{bc(g)}}. With the relationship of compound angle of orientation 603 solved through methods previously described the final unknown acceleration component 503 \vec{a_{pill(bc(g))}} can be solved by rotating the sensor pill measurement vector \vec{aₚᵢₗₗ} by the defined compound angles of orientation 603 to define the vector 512 \vec{aₚᵢₗₗ₋ᵣₒₜ} (shown in FIG. 7A).

As shown in FIG. 7A the sensor pill measurement vector rotated 512 \vec{aₚᵢₗₗ₋ᵣₒₜ} and the sensor patch measurement vector 510 \vec{a_{patch}} have common acceleration components due to: gravity 5101 and motion of the body core relative to gravity 5022. Further, the sensor patch measurement vector is entirely defined by the two acceleration component vectors. Therefore, the unknown acceleration component 5031 \vec{a_{pill-rot(bc(g))}} defining the sensor pill translation motion relative to the body core can be solved and defined by subtracting the sensor patch measurement vector 510 \vec{a_{patch}} from the sensor pill measurement vector rotated 512 \vec{aₚᵢₗₗ₋ᵣₒₜ} on the common coordinate system that is the sensor patch measurement coordinate system referenced to gravity.

\vec{aₚᵢₗₗ₋ᵣₒₜ}−\vec{a_{patch}}=\vec{a_{pill-rot(bc(g))}}

To characterize the motional activities of the lumens relative to the body core several condition concerning body core activity and lumen constraining condition on the pill have to be determined. For example, when the sensor pill is in a lumen of cross sectional area similar to sensor pill size dimensions, the acceleration component \vec{a_{pill(bc(g))}} sensor pill relative to the body core is also generally representative of the lumen movement relative to the body core. An example of this condition may be when the pill is in the small intestines. In this condition regardless of activity level of the body core the motional spatial translation effects as described by acceleration component \vec{a_{pill(bc(g))}} sensor pill relative to the body core provides a general description of lumen motional spatial translational response relative to the body core.

For the condition where the lumen cross sectional area is large compared with the sensor pill dimensions, such as the stomach, the sensor pill may have movement that is independent of the lumen movement when the body core is involved in rigorous activities. For example, during running or jogging, the sensor pill may bounce in the stomach and even be free of contact with the lumen walls. In this case the acceleration component \vec{a_{pill(bc(g))}} sensor pill relative to the body core, does not describe the motional relationship between the lumens and the body core because of the abrupt independent movement of the sensor pill relative to the lumen.

To further understand the conditional sets, first a summary review of the motional and orientation relationship attributes of the digestive track lumens to body core are covered. The lumens of the digestive track are attached to the body core abdominal cavity with mesentery tissue. The mesentery tissue attaches to the lumens and abdominal wall providing some support for the lumens relative to the body core. The mesentery web of tissue allows for movement of the lumens relative to the body core, however, the lumens are limited from significant orientation changes with respect to the body core. When the body core is at rest or experiencing minimal motional acceleration changes the lumens are held in a relatively constant relationship with the body core by the steady state of the gravitational vector. When the body core is active with rigorous activity such as jogging the lumens experience a related but different motional profile that is caused by momentum of the lumens and the non rigid loose characteristics of the mesentery web. Further, the difference in motional profiles between the body core and the lumens of the intestines are dominated by relative spatial movement and less by relative orientation changes. In summary, the physical limitations of the lumen in the digestive track due to the tissue called mesentery limit orientation change relative to the body core however, motional differences do occur relative to the body core. The motional differences between the lumens and the body core can be caused by the storage and release of energy of the mesentery tissues in response to rigorous activities experienced by the body core such as jogging.

Therefore, the motional translational relationships between the lumens and the body core can be achieved under certain condition. The orientation relationships of the pill can always be determined between the sensor pill and the body core. Further since the lumens have limited orientation changes with respect to the body core, faster rate orientation changes of the sensor pill to the body core can be interpreted as orientation changes of the sensor pill relative to the lumens.

The determination of whether the pill is located in a large or small lumen can be achieved by evaluation of the speed of the dynamic orientation changes of the pill relative to the body core. In summary, when the pill is constrained by a smaller lumen the dynamic orientation will be slower as compared to when the pill is tumbling in a larger lumen when the pill is unconstrained and may be bounced about by acceleration forces from body core motional activities.

Alternate embodiments of this invention include using multiple sensor pills simultaneously in the digestive track with a single sensor patch and single user interface processing unit as earlier describe. The advantages to launching multiple sensor pill into the digestive track allows for greater statistical characterization of localized areas of the digestive track. Further the pills may be different sizes and weights, and therefore different areas of the digestive track can be analyzed for tumbling effects associated with a given size and or weight of sensor pills.

In other embodiments, the system is modified to allow real-time orientation characteristics to be calculated within the pill with inputs from the sensor patch, and the pill further uses the real-time orientation information in conjunction with other sensor types within the pill such as optical imaging technologies. This type of analysis system uses the real time orientation and motional characteristics of the pill relative to the body core and in some condition the lumens to adjust data capture rates of the imaging sensors to provide improved image coverage of the lumens internal surface area while reducing power consumption to provide longer pill system operation for a given battery capacity.

Although specific embodiments of the invention have been disclosed, those having ordinary skill in the art will understand that changes can be made to the specific embodiments without departing form the spirit and scope of the invention. The scope of the invention is not to be restricted, therefore, to the specific embodiments. Furthermore, it is intended that the appended claims cover any and all such applications, modifications, and embodiments within the scope of the present invention.

I claim:

1) A measurement system comprising at least:
a) one or more in-vivo ingestible electronics sensing pill(s) that each encompasses electronic functions for at least: measuring three orthogonal axes of acceleration, and
b) one or more electronics sensing patch(s) that is for placement on the exterior surface of a body core and each encompasses electronic functions for at least: measuring three orthogonal axes of acceleration

2) A measurement and analysis system for characterizing an in-vivo device's dynamic acceleration translational motion and dynamic orientation relative to a body core while characterizing the body core's physical activity relative to gravity comprising at least:
a) one or more in-vivo ingestible electronics sensing pill(s) that encompasses electronic functions for at least: measuring three orthogonal axes of acceleration, and a method of wirelessly transmitting acceleration measurement data out of sensor pill as pill measurement data, and
b) one or more electronics sensing patch(s) that located on the exterior surface of a body core at a predetermined location with a predetermined orientation that encompasses electronic functions for at least: measuring three orthogonal axes of acceleration, and a method of wirelessly transmitting acceleration measurement data out of sensor patch as patch measurement data, and
c) a data processing user interface device that encompasses electronics for at least the following functions:
i) a method of wireless communication that can receive said pill measurement data and patch measurement data.
ii) digital memory for storage of said measurement data and storage of application software that comprises algorithms that provides for the interpretation of said measured data to define results timeline characterizing the sensor pill's dynamic motion and dynamics orientation relative to the body core and body core activities relative to gravity.
iii) a processor that can execute said software algorithms for the interpretation of said pill measurement data and patch measurement data to define results
iv) a method of conveying results to a person such as a display screen or another computer device such as a standard input-output port such as a USB port.

3) The measurement and analysis system recited in claim 2 with the method of wireless communication being implemented with radio wave signals.

4) The measurement and analysis system recited in claim 2 with the method of wireless communication being implemented with acoustic wave signals.

5) The measurement and analysis system recited in claim 2 with the said algorithms further provide results for:
a) characterizing of the lumen of the small intestines in terms of motional acceleration relative to the body core orientation and motion.
